(19) United States
(12) Patent Application Publication
(10) Pub. No.: US 2010/0190658 A1
(43) Pub. Date: Jul. 29, 2010

(54) COLORIMETRIC ASSAY FOR THE VISUAL DETECTION OF PRIMARY AND SECONDARY AMINES

(76) Inventors: Erik Van Der Eycken, Ninove (BE); Stijn Claerhout, Harelbeke (BE); Denis Ermolatev, Voronezh (RU); Eugene Babaev, Moscow (RU)

Correspondence Address: BACON & THOMAS, PLLC, 625 SLATERS LANE, FOURTH FLOOR, ALEXANDRIA, VA, 22314-1176, US

(21) Appl. No.: 12/667,505

(22) PCT Filed: Jul. 2, 2008
(86) PCT No.: PCT/EP2008/058537

§ 371 Date: Dec. 31, 2009

(30) Foreign Application Priority Data
Jul. 2, 2007 (GB) 0712844.0

(51) Int. Cl. C40B 30/04; G01N 33/00; C40B 40/04; G01N 33/544

(52) U.S. Cl. 506/9; 436/111; 506/15; 436/528

(57) ABSTRACT

The present invention concerns a novel method and kit system for the detection of solid-phase bound primary amines, secondary amines, or thiol groups comprising adding a fluid to said substrate and further comprising adding a novel reagents to said substrate and recording a colour reaction on said substrate that comprise said amine or said thiol groups. The method and kit of present invention can be used for the quantitative determination of organic substituents with primary or secondary amines or with thiol groups immobilized on or in insoluble materials.

### BACKGROUND OF THE INVENTION

A. Field of the Invention

The present invention relates generally to a new colorimetric assay for the visual detection or quantification of solid-phase bound primary and secondary amines, solid-phase bound thiols groups. Moreover it concerns the use a colorimetric assay in amines sensing.

The present invention also relates to a new colorimetric assay for the detection and/or quantification of substrate-bound primary amines and/or secondary amines and/or thiol groups. In addition, it also concerns the use of a colorimetric assay in sensing primary amines and/or secondary amines.

B. Technical Background

Several documents are cited throughout the text of this specification. Each of the documents herein (including any manufacturer's specifications, instructions etc.) are hereby incorporated by reference; however, there is no admission that any document cited is indeed prior art of the present invention.

Quantitative monitoring of resin bound amines is an important criterion for the quality control in peptide synthesis. Furthermore, biogenic amines are of widespread interest due to their importance in areas ranging from quality control of foodstuffs (F. Calbiani, et al. Rapid assay for analyzing biogenic amines in cheese: matrix solid-phase dispersion followed by liquid chromatography-electrospray-tandem mass spectrometry, (J. Agric. Food Chem. 53 (2005), pp. 3779-3783 and J. M. Landete, et al. Biogenic amines in wines from three Spanish regions, J. Agric. Food Chem. 53 (2005), pp. 1119-1124.) to biomarkers of disease (G. Preti, et al. Analysis of lung air from patients with bronchogenic carcinoma and controls using gas chromatography-mass spectrometry, J. Chromatogr. 432 (1988), pp. 1-11.) and (M. L. Simenhoff, et al. Biochemical profile or uremic breath, N. Engl. J. Med. 297 (1977), pp. 132-135). Thus, there is a clear need in the art for sensitive or simple assaying for amines and in particular to distinguish between resin with and without bound amines and that can monitor the resin binding. Hence, there is a clear need in the art for a sensitive and simple assay for the detection of primary amines and/or secondary amines that allows for monitoring the binding of primary amines and/or secondary amines to resins.

In the art a 2,4,6-trinitrobenzenesulfonic acid (TNBS) colour test is used to demonstrate resin bound (primary) amines. Present invention surprisingly found a new sensitive reagent and developed a very simple test for substrate bound primary and secondary amine that has a much higher sensitivity than the TNBS colour test towards primary amines. The test is also useful for assaying thiol groups.

### SUMMARY OF THE INVENTION

The present invention solves the problems of the related art of detecting substrate bound primary and secondary amine and sensing (biogenic) amines in synthetic or natural matrixes by providing a new sensitive reagent.

It was one object of the present invention to provide methods and devices for the accurate and convenient detection of primary amines and/or secondary amines and/or thiol groups.

It was one object of the present invention to provide a method for the accurate and convenient detection of solid-phase bound primary amines and/or secondary amines.

It was another object of the present invention to provide a method for the accurate and convenient detection of solid-phase bound thiol groups.

The methods and/or devices of the present invention allow for the detection of a broad scope of amines including in particular secondary amines that could not be detected with the TNBS test before. The method of the present invention is reliable and is used in a test that is easy to handle.

In accordance with the purpose of the invention, as embodied and broadly described herein, the invention is inter alia broadly drawn to a new colorimetric assay for the visual detection or quantification of solid-phase bound primary and secondary amines and thiols or for simple sensing of biogenic amines.

In accordance with the purpose of the invention, as embodied and broadly described herein, the invention is also broadly drawn to a new method and new devices for the visual detection or quantification of primary and secondary amines and thiols or for simple sensing of biogenic amines.

One aspect of the invention is a method for the detection of solid-phase bound primary amines, secondary amines, or thiol groups or for sensing biogenic amines comprising adding a fluid to said substrate and further comprising adding a detecting element which is a compound (formula 1) with basic structure

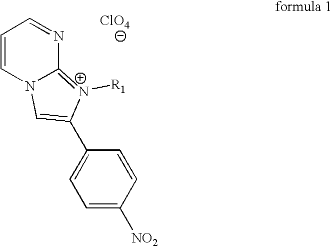

wherein is R¹ is a group selected of Me, i-Pr, Cyclododecyl, i-Bu, Cyclohexyl, Cyclopropyl, Bu, Hex, Et, Bn and Veratryl and to said substrate and recording a colour reaction on said substrate that comprise said amine or said thiol groups.

In a preferred embodiment, the fluid is selected from the group consisting of DMF (dimethylformamide), DMSO (dimethylsulfoxide), acetonitrile, methanol, water, acetone and mixtures of the aforementioned fluids.

In an embodiment of the present invention, R¹ is selected from the group consisting of methyl, isopropyl, cyclododecyl, isobutyl, cyclohexyl, cyclopropyl, butyl, hexyl, ethyl, benzyl and veratryl, preferably R¹ is methyl.

In another embodiment R¹ represents

a linear or branched, saturated or unsaturated, unsubstituted or at least mono-substituted aliphatic radical;

or an unsubstituted or at least mono-substituted aryl, which may be bonded via a linear or branched alkylene, alkenylene or alkinylene group.

In yet another embodiment R¹ represents

an unsubstituted or at least mono-substituted linear or branched C₁₋₁₀ alkyl radical, C₂₋₁₀ alkenyl radical or C₂₋₁₀ alkinyl radical;

or an unsubstituted or at least mono-substituted 6-, 10- or 14-membered aryl, which may be bonded via a linear or branched, unsubstituted or at least mono-substituted C₁₋₆ alkylene, C₂₋₆ alkenylene or C₂₋₆ alkinylene group.

Preferably the aforementioned C₁₋₁₀ alkyl radical, C₂₋₁₀ alkenyl radical or C₂₋₁₀ alkinyl radicals may in each case be unsubstituted or optionally substituted with 1, 2, 3, 4 or 5 substituent(s) independently selected from the group consisting of F, Cl, Br, I, —CN, —CF₃, —OCF₃, —SCF₃, —OH, —SH, —NH₂, —O—C₁₋₅-alkyl, —S—C₁₋₅-alkyl, —NH(C₁₋₅-alkyl) and —N(C₁₋₅-alkyl)₂.

Preferably the aforementioned C₁₋₆ alkylene, C₂₋₆ alkenylene or C₂₋₆ alkinylene groups may in each case be unsubstituted or substituted with 1, 2 or 3 substituent(s) independently selected from the group consisting of F, Cl, Br, I, —CN, —CF₃, —OCF₃, —SCF₃, —OH, —SH, —NH₂, —O—C₁₋₅-alkyl, —S—C₁₋₅-alkyl, —NH(C₁₋₅-alkyl) and —N(C₁₋₅-alkyl)₂.

Preferably the 6-, 10- or 14-membered aryl may be unsubstituted or optionally substituted with 1, 2, 3, 4 or 5 substituent(s) independently selected from the group consisting of C₁₋₅-alkyl, —O—C₁₋₅-alkyl, —S—C₁₋₅-alkyl, —C(═O)—OH, —C(═O)—C₁₋₅-alkyl, —C(=O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenoxy and benzyl.

Another aspect of the present invention is a method for the detection of a substrate-bonded compounds containing at least one substituent selected from the group consisting of primary amino-groups, secondary amino-groups and thiol-groups, comprising the steps of:

adding a fluid to the substrate;

adding a reagent comprising a compound of general formula 1

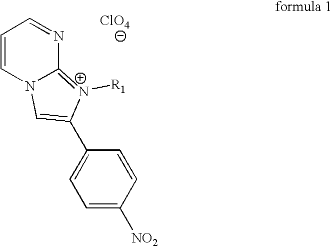

wherein R¹ is selected from the group consisting of methyl, isopropyl, cyclododecyl, isobutyl, cyclohexyl, cyclopropyl, n-butyl, hexyl, ethyl, benzyl and 3,4-dimethoxy-benzyl alcohol to the substrate and

recording a colour change on the substrate.

Preferably the fluid is added to the substrate at a temperature in the range between 10° C. and 30° C., more preferably at a temperature in the range between 15° C. and 25° C.

Preferably the reagent is added to the substrate at a temperature in the range between 15° C. and 80° C., more preferably at a temperature in the range between 25° C. and 60° C., even more preferably at a temperature in the range between 50° C. and 60° C.

Preferably a colour change is detected 10 minutes after adding the reagent to the substrate, more preferably 5 minutes after adding the reagent to the substrate, even more preferably between 1 minute and 5 minutes after adding the reagent to the substrate.

These secondary amino groups, the primary amino groups or the thiol groups can be in or on a substrate or the amino or thiol group can be on an organic substituent bound to said substrate or entrapped in said substrate.

In the sense of the present invention an organic substituent is an organic compound.

In a particular embodiment of present invention the organic substituents are peptides or peptide-based molecules. The peptides or peptide-based molecules contain primary amino and/or secondary amino and/or thiol groups.

In another particular embodiment of the present invention the organic substituent is an amino acid. Preferably the amino acid is an amino acid selected from the group consisting of isoleucine, leucine, lysine, methionine, phenylalanine, threonine, tryptophan, valine, alanine, asparagine, aspartic acid, carnitine, citrulline, cysteine, cystine, GABA, glutamic acid, glutamine, gluthathione, glycine, hydroxyproline, ornithine, proline, serine, taurine, and tyrosine.

In another particular embodiment of the present invention the organic substituent is an antiobiotic.

Such organic substituents are spread over a substrate as a substituents array.

The substituents which comprise at least one primary amino group, at least one secondary amino group or at least one thiol group are entrapped in said substrate or such substituents may be entrapped in polymeric microspheres.

In another embodiment of the present invention the substrate is an animal tissue or plant tissue.

In yet another embodiment of the present invention the substrate is collageneous, cartilagenous material or bone.

Another aspect of the invention is that the amino groups or the thiol groups are quantified based on the colour reaction. The amino groups or the thiol groups can be quantified based on the darkness of the colour or on a corresponding greyscale and are convertible into a corresponding amine number or thiol number, for instance they are expressible as mmol/g of solids.

In another aspect of the present invention, the amount of primary amino groups and/or secondary amino groups and/or thiol groups that are in each case optionally bound to a substrate are quantified based on the colour reaction. The amount of primary amino groups and/or secondary amino groups and/or thiol groups that are in each case optionally bound to a substrate can be quantified based on the darkness of the colour or on a corresponding greyscale and are convertible into a corresponding amine number and/or thiol number, for instance the amount can be expressed as mmol/g of solids.

The colour reaction is equivalent to a colour change. The intensity of the colour change is proportional to the total concentration of amino groups and/or thiol groups, providing a qualitive analysis for amine content and/or thiol content.

The colour change may be detected through visual observation, under optical microscope, or by spectroscopic means. In particular, when using visible light with a wave length between 380 nm and 780 nm for detection, a compound with amino groups and/or thiol groups that was reacted with a DESC reagent of formula 1 shows an absorption spectrum that is different from the unreacted compound. For example, piperidine that was reacted with a DESC reagent of formula 1 shows a strong absorption at 455 nm and 361 nm.

The colour change may be any type of change. For example, the change may be colouring, decoloration, colour tone change or the like. The change may also be a spectral change observable in an ultraviolet range or a near infrared ray range. The colour change may be a sole change or a combination of two or more changes. The change may be a colour change detectable with any one of various measurement techniques available to those skilled in the art, absorbance measurement of a reflected or transmitted ultraviolet ray or visible ray, microscopic or visual observation thereof and so forth, or detectable with any combination of two or more of these measurement techniques.

In a preferred embodiment the colour change is from uncoloured to a yellow-red colour.

Still another aspect of the present invention is a kit comprising the DESC reagent (the reagent comprising the compound of formula 1)

and instructions for using the compound in an assay to determine secondary amino groups, primary amino groups or thiol groups in or on a substrate. Such substrate can be at least one solid particle. The substrate may be at least one bead. Preferably, the beads are made of amine-modified polystyrene.

Or the substrate may be selected of the group consisting of a resin, a polymer. Examples of suitable resins include aminomethylated polystyrene resin with a loading of 0.2 mmol/g, 0.5 mmol/g or 0.9 mmol/g and Hypogel® with a loading of 0.9 mmol/g.

In yet another aspect of the invention the substrate comprises latex beads, nanoparticles, macro-beads, membranes, microplates, array surfaces and dipsticks. Such substrate may be amorphous, crystalline, macroporous or microporous.

In another embodiment of present invention the detecting element (a compound with basic structure (formula 1) herein also called DESC reagent) can be in or on a substrate and reacts in a colour reaction when amines are extracted to or absorbed in said substrate. The substrate may be at least one bead. Or the substrate may be selected of the group consisting of a resin, a polymer. In yet another aspect of the invention the substrate comprises latex beads, nanoparticles, macro-beads, membranes, microplates, array surfaces and dipsticks. Such substrate may be amorphous, crystalline, macroporous or microporous.

In another embodiment, examples of suitable substrates include polystyrene, polystyrene/polyethylene glycol graft copolymer, silica gel, glass beads, controlled pore glass, agarose, sepharose, a solid polymer having a primary amine, a solid polymer having a secondary amine, cellulose, polypropylene, polyurethane, chitosan, polyacrylonitrile, polysulfone, polymethacrylate, polyacrylamide, polyvinyl alcohol and modified derivatives thereof.

In another embodiment of present invention, the DESC test or DESC reagent is used to indicate or quantify the presence of an amount of amines, biogenic amines, free amino acids, secondary amino groups, primary amino groups or thiol groups and to transfer this in a DESC-value by measuring the colour of the yellow, orange or red compounds formed, for instance by using a spectrophotometer or photo-electric cells for measuring colour temperature. Systems for measuring colour are available for the skilled man and have been described in handbooks (Handbook of Colour Science [Second Edition]. Edited by The Colour Science Association of Japan, Published from University of Tokyo Press, 1538 pp., June 1998, 7-3-1 Hongo, Bunkyo-ku, Tokyo 113-8654, Japan and Measuring Colour, Dr. R. W. G. Hunt, 3rd Edition, Fountain Press, Surrey, United Kingdom, 336 pp., 1998).

Amines are widely found in nature. For example, biogenic amines are present in living cells and in food products. A number of naturally occurring primary and secondary amines can be found in meat and fish as it spoils (e.g., histamine, putrescine, cadaverine, methyl amine), tobacco smoke (e.g., methylamine, dimethylamine, pyrrolidine), and beer (e.g., ethylamine, isoamylamine, dibutylamine). Other naturally occurring, non-chromophoric amines include glucosamine, galactosamine, mannosamine, and heparin. Although amines are generally easy to separate, the lack of a chromophore in their structure makes amines difficult to detect.

The thiol group-containing compounds according to the present invention are not particularly limited, and any compounds having one or more thiol groups may be measured. Examples of the thiol group-containing compound include, but not limited thereto, alkylthiols (for example, methylmercaptan, ethylmercaptan and the like) arylthiols (for example, thiophenol, thionaphthalene, benzylmercaptan and the like), amino acids or derivatives thereof (for example, cysteine, glutathione and the like), peptide compounds (for example, cysteine residue-containing dipeptide compounds, tripeptide compounds, tetrapeptide compounds, oligopeptide compounds containing five or more amino acid residues and the like), proteins (for example, globular proteins in which cysteine residues are exposed on their surfaces and the like) and so forth.

Identification or quantification of amines, biogenic amines, free amino acids, compounds with secondary amino groups, compounds with primary amino groups or compounds with thiol groups in a mixture can be quick in a TLC (thin layer chromatography) procedure. Moreover the specific compound comprising secondary amino groups, primary amino groups or thiol groups can by this means be identified in a mixture when the R_{f} of a compound is compared with the R_{f} of a known compound (preferably both run on the same TLC plate). Suitable TLC plates are sheets of glass, metal, or plastic which can be coated with a thin layer of a solid adsorbent (usually silica or alumina). A small amount of the mixture to be analyzed is spotted near the bottom of this plate. The TLC plate can then be placed in a shallow pool of a solvent in a developing chamber so that only the very bottom of the plate is in the liquid comprising the mixtures. This liquid, or the eluent, is the mobile phase, and it slowly rises up the TLC plate by capillary action. When the solvent has reached the top of the plate, the plate is removed from the developing chamber, dried, and the separated components of the mixture are visualized. If the compounds on the TLC plate comprises secondary amino groups, primary amino groups or thiol groups, these can be colored by the DESC reagent of present invention.

Another aspect of present invention is an optical sensor that indicates solution phase or gas phase amine species. Such sensors can be achieved by incorporation of the DESC reagent (formula 1) of present invention within ultra thin polymeric films (e.g. <0.5 μm) which for instance can be deposited via spin coating on a planar fused silica waveguide. Extraction of amines or compounds with amino groups into the films will result in a colour change. The use of the waveguide sensing configuration enables very fast response times without loss in sensitivity since the optical pathlength is significant (e.g., absorbance is enhanced about 20 times even when 0.3 μm thick film is employed).

The optical sensing technology for sensitive detection of amine vapours can be also a microsphere sensor based on the DESC reagent being adsorbed onto a silica sphere matrix. When the amines adsorb onto or into the matrix, a colour will be formed from yellow, orange to dark red. The colour change can be detected with a fiber optic spectrometer.

A fast response times of such sensors makes such devices potentially useful as detectors in flow-through analytical systems such as flow-injection analysis (FIA) or gas/liquid phase chromatography. Such tin plate sensors may also be particularly suitable for detecting amine vapours or volatile compounds with secondary amino groups, primary amino groups or thiol groups.

Present invention in a particular embodiment also concerns a sensor with the sensing element (DESC reagent (formula 1) of present invention) for the detecting of analytes of the group consisting of amines, biogenic amines, free amino acids, compounds with secondary amino groups, compounds with primary amino groups or compounds with thiol, being immobilized in carbon nanotubes and eventually embedded in a polymer.

In another embodiment of the present invention, the compounds of general formula 1 are utilized in the preparation of a disposable sensor for the detection of spoilage in package raw fish, meat, and poultry for consumer applications. The sensor includes a thin circular wafer which is placed in direct contact with the food of interest before the final wrapping is put in place. In the absence of amines and/or thiol-groups, the wafer remains colourless indicating that the food is safe to consume. However, when amines and/or thiol-groups are present, a colour change takes place, indicating that the food is no longer safe for consumption.

The interface with the raw fish or meat product is a porous membrane having a pre-determined molecular weight cutoff which permits small molecules such as amines to pass, but will not allow intact cells, proteins, or other contaminants to pass.

Suitable examples of the porous membrane include cellulose, cellulose acetate, polypropylene, polyurethane, polyacrylonitrile, nitrocellulose, polysulfone, polyacrylamide, polymethacrylate, polyamide and modified derivatives thereof.

A second layer, which is located adjacent to the porous membrane, is formed of a porous support to which the compounds of general formula 1 are bonded. Suitable examples of supports include cellulose, polypropylene, polyurethane, chitosan, polyacrylonitrile, polysulfone, polyvinyl alcohol, agarose, sepharose, polymethacrylate, polyacrylamide, polystyrene, polystyrene/polyethylene glycol graft copolymers, silica gels, glass beads, controlled pore glass and modified derivatives thereof.

The wafer may also include additional layers such as a suitable diffusable layer which may serve as a visible surface of the device as well as for the potential entrapment of co-reagents if necessary. In addition, the wafer may also include an opaque top layer which is utilized to modify the design. The wafer is assembled with an adhesive. After placement on the raw fish or meat surface, packaging material is placed over the wafer to hold the wafer in place on the surface of the meat product by pressure. Alternatively, an adhesive may be applied to the outer perimeter of the top exposed surface of the wafer to adhere it to the packaging material.

The present invention also concerns a sensing/emitting layer comprising the DESC reagent which will changes colour upon exposure to the analyte liquids or vapours with analytes of the group consisting of amines, biogenic amines, free amino acids, compounds with secondary amino groups, compounds with primary amino groups or compounds with thiol groups.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DETAILED DESCRIPTION

### DETAILED DESCRIPTION OF EMBODIMENTS OF THE INVENTION

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Referring now specifically to the drawings, a colour test for identifying and quantifying of solid-phase bound primary amines, secondary amines and thiol groups according to an embodiment of the present invention is illustrated in FIG. 4, FIG. 5, FIG. 7 and FIG. 9. For practical reasons in the figures of this application the colours varying from white, yellow, orange to red are presented in grey scale. The colour test system of present invention has been demonstrated to be more sensitive than the TNBS colour test of the art.

Definitions

TNBS is 2,4,6-trinitrobenzenesulfonic acid and the TNBS colour test has for instance been developed and described by Means, G. E. and Feeney, R. E. 1971. (Akylating and similar reagents. In Chemical modification of proteins. Holden-Day Press. pp. 130-132, 144-148, 216-217, 222-223. San Francisco, Calif.) and is currently been available at NovaBiochem (see NovaBiochem catalogue for protocol).

### Examples

### Example 1

The development of the “DESC” test is the result of the search for a new synthetic approach for the synthesis of fully substituted 2-aminoimidazoles of which the syntheses has been demonstrated in FIG. 1. Desc can be produced according to the method of Smirnova et al, 2003 (Smirnova, T. A.; Kurapov, P. B.; Vetrova, E. L.; Bass, A. G.; Nam, N. L.; in Kafedra Org. Khim., Timiryazevsk. S-Kh. Akad., Russia. Izvestiya Timiryazevskoi Sel'skokhozyaistvennoi Akademii (2003), (4), 132-141) and its bromide analoges can be produced according to the method of Ermolat'ev et al. 2006 (Efficient One-pot two-step microwave-assisted procedure for the synthesis of polysubstituted 2-aminoimidazoles, D. S. Ermolat'ev, E. V. Babaev, E. Van der Eycken, Org. Lett., 8, 5781-5784, 2006). Some of the 1-alkyl-2-aryl-imidazo[1,2-a]pyrimidinium perchlorates, which are intermediates for the synthesis of substituted 2-aminoimidazoles as demonstrated in FIG. 1, seemed to give a strong colour change when reacted with primary as with secondary amines. This was tested for resin bound amines and this gave very clear colour change of the resin beads.

1.1. Visual Comparison of the DESC Test with the TNBS Test at Different Sensitivity Levels of the Primary Amine and Testing Procedure for Secondary and Primary Amines.

A series of resin samples of known free amine content was prepared by treatment of aminomethylated polystyrene resin with a mixture of Fmoc-Ala (Fmoc: 9-fluorenylmethoxycarbonyl) and Boc-Ala (Boc: tert-butoxycarbonyl group) in varying proportions. After selective removal of the Fmoc-protective group beads were obtained with a free amine content of 100, 50, 20, 10, 5, 2 and 1%. The different fractions were treated with both DESC and TNBS (FIG. 4). Also an on resin secondary amine was reacted with the DESC and TNBS test, this is shown in entry 8 of FIG. 4. The structure of the resin bound secondary amine is given in FIG. 3. The sensitivity of the DESC molecule towards primary amines is given in FIG. 4.

The following procedure has been developed for monitoring the completeness of a peptide coupling reaction on solid support, for the detection of primary amines. A few beads are suspended in 100 μL of a 5% DESC solution in DMF (dimethylformamide). After heating at 70° C. during 5 minutes, the beads are washed with DMF (3×), MeOH (3×) (methanol) and DCM (3×) (dichloromethane). Beads containing free primary amino groups appear as red spheres while completely coupled beads

(containing no free amino groups) remain colourless. For the detection of secondary amines the procedure is slightly different. A few drops of a 20% DIPEA (diisopropylethylamine) solution in DMF should be added after addition of the solution comprising the DESC reagent.

The test solution colours dark after addition of the DIPEA solution, but after washing (according the procedure described above), the beads containing free secondary amine colour red while beads containing no secondary amine remain colourless.

A photograph (FIG. 5) is provided of part of a resin mixture containing beads with 5% free amino groups and beads without amino group after treatment with TNBS (left picture of FIG. 5) and DESC (right picture of FIG. 5). A mixture of totally protected resin and resin containing 5% free primary amino groups (synthesized according to the method described earlier) was mixed and reacted with the TNBS and the DESC test. The resin used for this was aminomethylated polystyrene resin with a loading of 0.5 mmol/g.

So far, solid phases that have been used include Hypogel-NH₂ resin (0.92 mmol/g) and aminomethylated polystyrene resin (0.5 mmol/g and 0.9 mmol/g).

1.2. Test at Different Sensitivity Levels of the Secondary Amine.

In FIG. 4, entry 8, we can see that the DESC-test also gives a strong colour change with secondary amines. Therefore, we decided to test the sensitivity of the DESC-test on secondary amines.

A series of resin samples of known free amine content was prepared by treatment of aminomethylated polystyrene resin with a mixture of Fmoc-Ala and Boc-Ala in varying proportions. After selective removal of the Fmoc-protective group the resin was reacted with a backbone amide linker (BAL), by which an aldehyde moiety was introduced. Reductive aminiation of the aldehyde group with phenylalanine allylester gave beads with a free amine content of 100, 50, 20, 10, 5, and 2% of the secondary amine (FIG. 6). The sensitivity of the DESC molecule towards secondary amines is given in FIG. 7.

### Example 2

The DESC-test was also tested on a resin bound thiol. This seemed to give a strong colour change of the resin (FIG. 9). The structure of the resin bound thiol is given in FIG. 8; it is derived of a trityl deprotected commercially available resin with a loading of 0.88 mmol/g. The sensitivity of the test on resin bound thiols was not determined but the strong colour change with 100% loaded resin suggest that this test can also be used to determine the completeness of the coupling to resin bound thiols (FIG. 9).

2.1 Solubility and Stability of the DESC Molecule.

The DESC reagent has good solubility in DMF, acetonitrile, methanol and aceton. It is not soluble in dichloromethane, even not after heating.

A solution of DESC was stored at room temperature for one month and no decomposition occurred. The test was still reliable after storage of the DESC solution for several months at room temperature. The DESC molecule is also stable at room temperature for several months.

The stability of the product was tested with TLC after several months and gave pure product after every test.

To guarantee the reliability of the DESC-test, it is recommended to refresh the DESC solution monthly, although tests have proved the stability of the solution for several months.

2.2. Test of the Influence of the Substitution of the 1-Alkyl-2-aryl-imidazo[1,2-a]pyrimidinium perchlorates on the Colour Change when Reacted with Resin Bound Primary Amines.

Several 1-alkyl-2-aryl-imidazo[1,2-a]pyrimidinium perchlorates were tested to see which ones colour resin bound primary amines. The results are given in FIG. 10. All the salts containing a 4′-nitro group at R₂ gave red colour change of the resin beads. Variation at R₁ gave no influence on the colour change (FIG. 10).

It will be apparent to those skilled in the art that various modifications and variations can be made in the concentration of the colouring agent and the substrate using compounds of the present invention and in construction of the system and method without departing from the scope or spirit of the invention. Examples of such modifications have been previously provided.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 demonstrates the synthesis of substituted 2-aminoimidazoles.

FIG. 2 shows the structure of the molecule DESC that can be used for the colour test.

FIG. 3 provides the structure of the resin bound secondary amine, wherein the resin is aminomethylated polystyrene with a loading of 0.5 mmol/g (NOVABIOCHEM®, Merck KGaA, Darmstadt, Germany)

FIG. 4 is a schematic view showing different sensitivity levels of the primary amine and one sensitivity level of the secondary amine. DESC or TNBS provides colour reaction in a yellow—orange to red that correlates to the levels of free amines. In the photographs, the colour scale is converted in a greyscale. The darkness of the beads positively relates to the content of the amines (primary or secondary amine are tested).

When using the same amount of primary amine and/or secondary amine and/or thiol groups the DESC test leads to a stronger and more intense colour change in comparison to the TNBS test. As the DESC test is more sensitive towards detecting primary amines and secondary amines and thiol groups a lower amount of amine and thiols, respectively, compared to the TNBS test can be detected. In particular, also small amounts of amines and thiol groups can be detected in a short time.

FIG. 5 is a photograph of part of a resin mixture containing beads with 5% free primary amino groups and beads without amino group after treatment with TNBS (left picture) and DESC (right picture). Colour reaction is on the photograph displayed by a greyscale and beads with 5% free amino groups are darker than beads with no free amino groups.

FIG. 6 demonstrates the synthesis of different levels of secondary amine.

FIG. 7 demonstrates the sensitivity test with different levels of secondary amine.

FIG. 8 displays the structure of the resin bound thiol.

FIG. 9 is a photograph demonstrating resin bound thiol with DESC-test.

FIG. 10 provided a tabulated overview of several 1-Alkyl-2-aryl-imidazo[1,2-a]pyrimidinium perchlorates which were tested to see which ones colour resin bound primary amines.

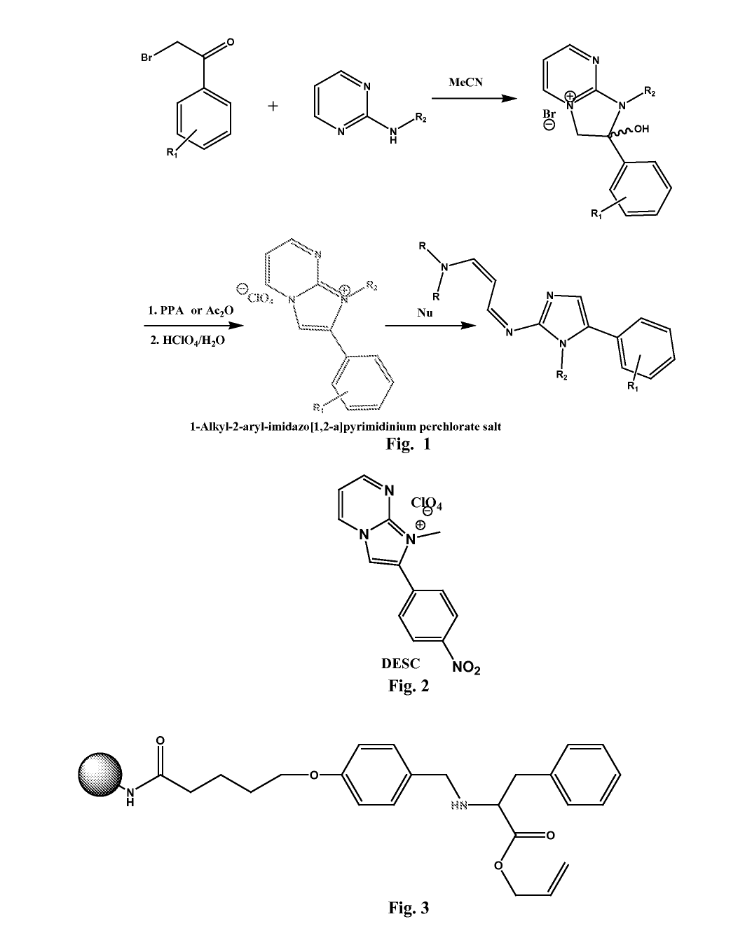

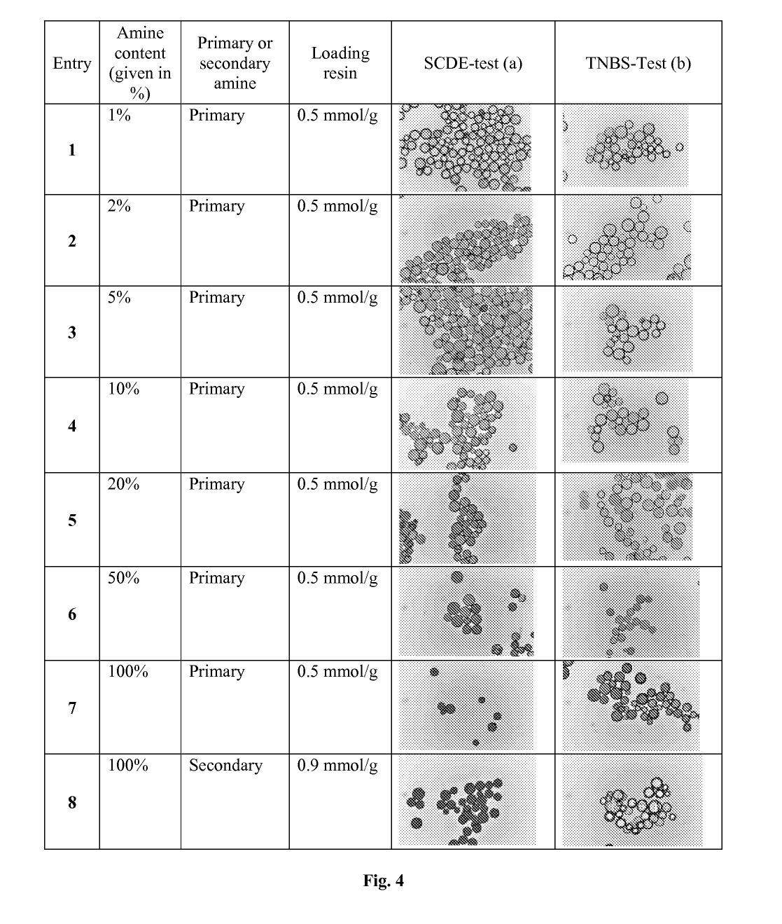

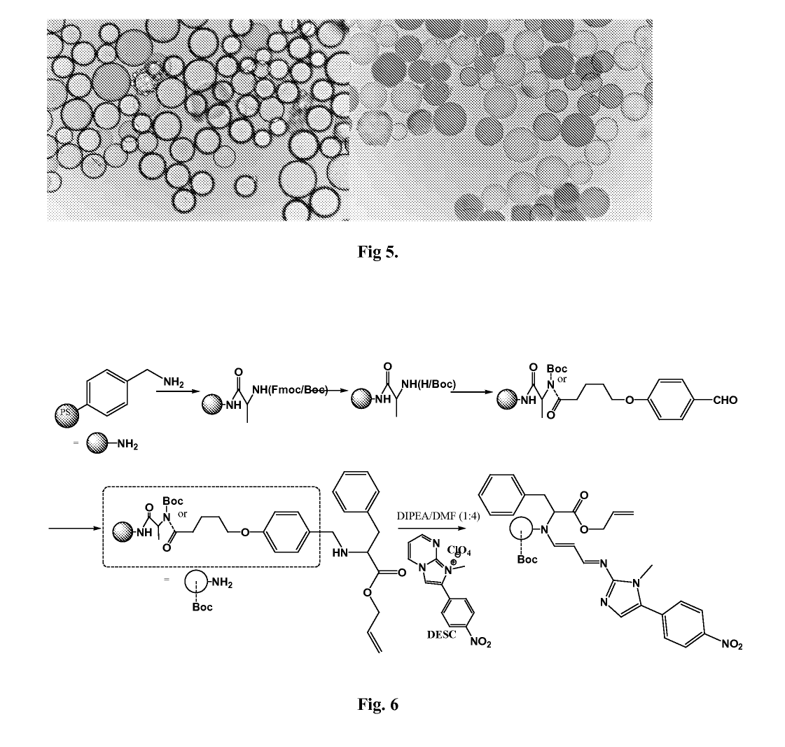

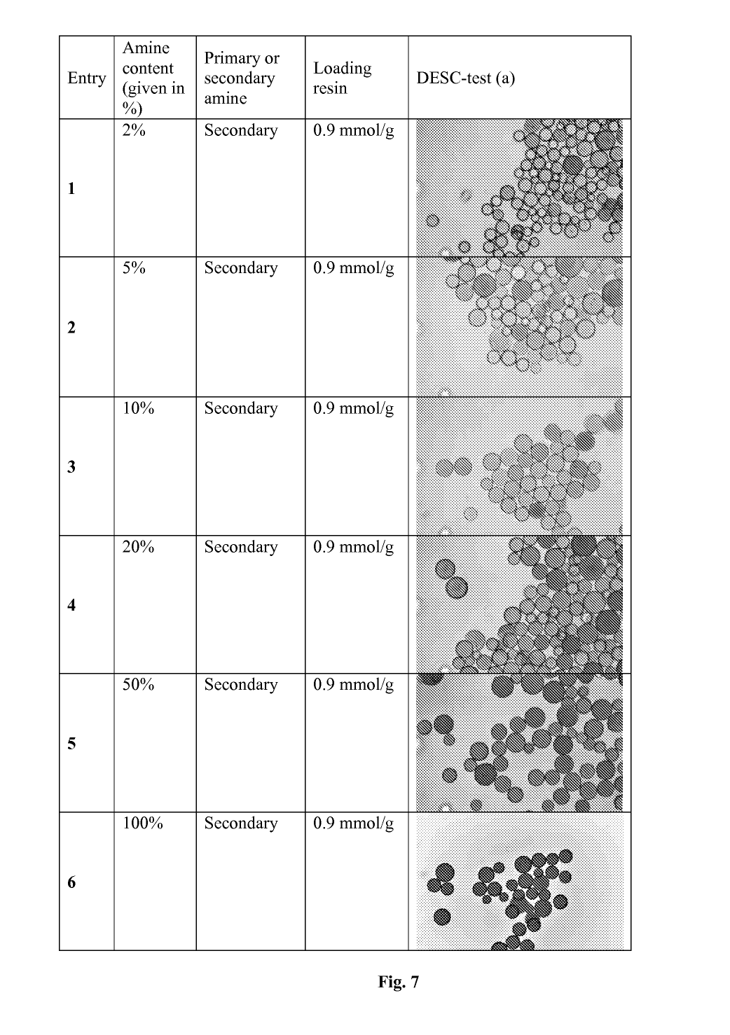

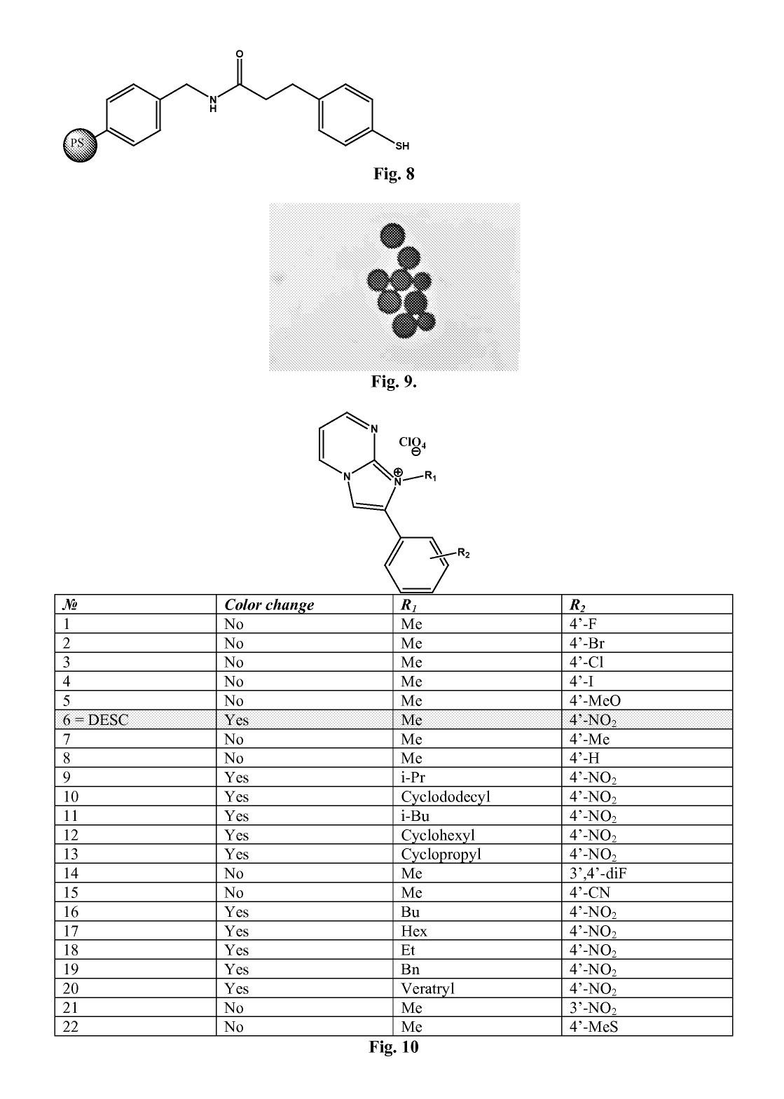

1-41. (canceled)

42. A method for the detection of solid-phase bound primary amines, secondary amines, or thiol groups comprising adding a fluid to said substrate and further comprising adding a reagent comprising a compound (formula I) with basis structure 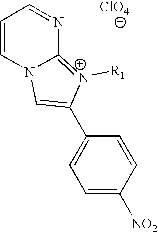
wherein R1 is a group selected of Me, i-Pr, Cyclododecyl, i-Bu, Cyclohexyl, Cyclopropyl, Bu, Hex, Et, Bn and Veratryl to said substrate and recording a colour reaction on said substrate that comprise said amino or said thiol groups.

43. The method according to claim 42, wherein the reagent compound has the following structure

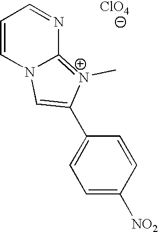

44. The method according to claim 42, wherein the secondary amino groups, the primary amino groups, the thiol groups is in or on a substrate.

45. The method according to claim 42, wherein the amino groups, or the thiol groups are quantified based on the colour reaction.

46. The method according to claim 42, wherein the amino groups, the thiol groups are quantified based on the darkness of the colour or on a corresponding greyscale and are convertible into a corresponding amine number or thiol number, for instance they are expressible as mmol/g of solids.

47. The method according to claim 42, wherein the amino groups, the thiol groups are on organic substituents bound to said substrate.

48. The method according to claim 42, wherein the organic substituents are peptides or peptide-based molecules.

49. The method according to claim 42, wherein the organic substituent is an amino acid.

50. The method according to claim 42, wherein the organic substituent is an antibiotic.

51. The method according to claim 42, wherein the organic substituents are spread over a substrate as an organic substituents array.

52. The method according to claim 42, wherein the substrate is at least one solid particle.

53. The method according to claim 42, wherein the substrate is at least one bead.

54. The method according to claim 42, wherein said substrate is a resin.

55. The method according to claim 42, wherein the substrate is a polymer.

56. The method according to claim 42, wherein the substrate is an animal tissue or plant tissue.

57. The method according to claim 42, wherein the substrate is collagenous, cartilaginous material or bone.

58. The method according to claim 42, wherein the substrate is amorphous.

59. The method according to claim 42, wherein the substrate is crystalline.

60. The method according to claim 42, wherein the substrate is macroporous.

61. The method according to claim 42, wherein the substrate is microporous.

62. The method according to claim 42, wherein the substrate is selected of the group consisting of latex beads, nanoparticles, macro-beads, membranes, microplates, array surfaces and dipsticks.

63. The method according to claim 42, wherein the organic substituents which comprise at least one primary amino group, at least one secondary amino group or at least one thiol group are entrapped in said substrate.

64. The method according to claim 42, wherein the organic substituents which comprise at least one primary amino group, at least one secondary amino group or at least one thiol group are entrapped in polymeric microspheres.

65. A kit comprising a compound of formula (I) with basis structure 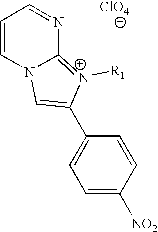
wherein R1 is a group selected of Me, i-Pr, Cyclododecyl, i-Bu, Cyclohexyl, Cyclopropyl, Bu, Hex, Et, Bn and Veratryl to said substrate and recording a colour reaction on said substrate that comprise said amino or said thiol groups and instructions for using the compound in an assay to determine secondary amino groups, primary amino groups, or thiol groups in or on a substrate.

66. A process for using a method for the detection of solid-phase bound primary amines, secondary amines, or thiol groups comprising adding a fluid to said substrate and further comprising adding a reagent comprising a compound (formula I) with basis structure 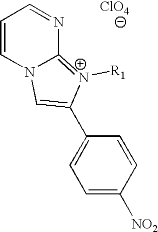
wherein R1 is a group selected of Me, i-Pr, Cyclododecyl, i-Bu, Cyclohexyl, Cyclopropyl, Bu, Hex, Et, Bn and Veratryl to said substrate and recording a colour reaction on said substrate that comprise said amino or said thiol groups, or the kit comprising a compound of formula (I) with basis structure 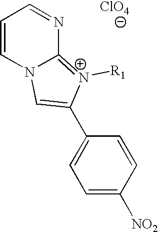
wherein R1 is a group selected of Me, i-Pr, Cyclododecyl, i-Bu, Cyclohexyl, Cyclopropyl, Bu, Hex, Et, Bn and Veratryl to said substrate and recording a colour reaction on said substrate that comprise said amino or said thiol groups, and instructions for using the compound in an assay to determine secondary amino groups, primary amino groups, or thiol groups in or on a substrate, for the quantitative determination of organic substituents with primary or secondary amines or with thiol groups immobilized on or in insoluble materials

67. An optical sensing system to indicate or quantify the presence of an amount of selected analyte, wherein the optical sensing system comprises a detecting element which is immobilized in or on a matrix of the group consisting of a carbon nanotubes matrix, a film, a sticker, a sheet of glass, a sheet of metal, a sheet of plastic, an ultra thin polymeric film, a microsphere matrix and a tin plate, and wherein the detecting element is a reagent comprising a compound of formule 1 with basis structure 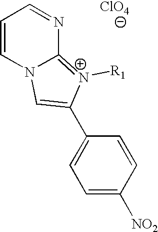
wherein R1 is a group selected of Me, i-Pr, Cyclododecyl, i-Bu, Cyclohexyl, Cyclopropyl, Bu, Hex, Et, Bn and Veratryl to said substrate and recording a colour reaction on said substrate that comprise said amino or said thiol groups, to form a colour reaction with the analyte of the group consisting of amines, biogenic amines, free amino acids, compounds with a least one secondary amino group, compounds with at least one primary amino group, or compounds with at least one thiol.

68. The optical sensing system according to claim 67, wherein the matrix is further embedded in a second layer.

69. The optical sensing system according to claim 68, wherein the second layer is a polymer.

70. The optical sensing system according to claim 67, wherein the analytes are liquid analytes.

71. The optical sensing system according to claim 67, wherein the analytes are vapour analytes.

72. The optical sensing system according to claim 67, further comprising a measuring means for measuring colour temperature.

73. The optical sensing system of according to claim 67, wherein the measuring means comprises a means of the group consisting of a spectrophotometer, a photo-electric cell and a fiber optic spectrometer.

74. The optical sensing system according to claim 67, further comprising a means for extracting or absorbing the analyte into matrix with the detecting element to achieve the colour change.

75. The optical sensing system according to claim 67 for use in a flow-through analytical system for instance a gas/liquid phase chromatography.

76. A method for the detection of a compound containing at least one substituent selected from the group consisting of primary amino-groups, secondary amino-groups and thiol-groups, comprising the steps of:
adding a reagent comprising a compound of general formula 1 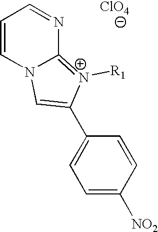
wherein R1 is an unsubstituted or at least mono-substituted linear or branched C₁₋₁₀ alkyl radical, C₂₋₁₀ alkenyl radical or C₂₋₁₀ alkinyl radical; or an unsubstituted or at least mono-substituted 6-, 10- or 14-membered aryl, which may be bonded via a linear or branched, unsubstituted or at least mono-substituted C₁₋₆ alkylene, C₂₋₆ alkenylene or C₂₋₆ alkynylene group and
recording a colour change on the substrate.

77. The method according to claim 76, wherein the compounds containing at least one substituent selected from the group consisting of primary amino-groups, secondary amino-groups and thiol-groups is a peptide, peptide-based molecule or amino acid.

78. The method according to claim 76, wherein the compounds containing at least one substituent selected from the group consisting of primary amino-groups, secondary amino-groups and thiol-groups are bound to a solid particle.

79. The method according to claim 78, wherein the solid particle is made of polystyrene, polystyrene/polyethylene glycol graft copolymer, silica gel, glass beads, controlled pore glass, agarose, sepharose, a solid polymer having a primary amine, a solid polymer having a secondary amine, cellulose, polypropylene, polyurethane, chitosan, polyacrylonitrile, polysulfone, polymethacrylate, polyacrylamide, polyvinyl alcohol, modified derivatives thereof or mixtures thereof.

80. The method according to claim 76, wherein R1 is methyl.

81. A sensor for the detection of a compound containing at least one substituent selected from the group consisting of primary amino-groups, secondary amino-groups and thiol-groups comprising a compound of general formula 1 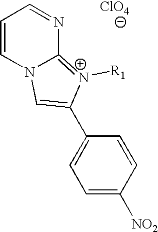
wherein R1 is an unsubstituted or at least mono-substituted linear or branched C₁₋₁₀ alkyl radical, C₂₋₁₀ alkenyl radical or C₂₋₁₀ alkynyl radical; or an unsubstituted or at least mono-substituted 6-, 10- or 14-membered aryl, which may be bonded via a linear or branched, unsubstituted or at least mono-substituted C₁₋₆ alkylene, C₂₋₆ alkenylene or C₂₋₆ alkynylene group.

82. A process using a sensor for the detection of a compound containing at least one substituent selected from the group consisting of primary amino-groups, secondary amino-groups and thiol-groups comprising a compound of general formula 1 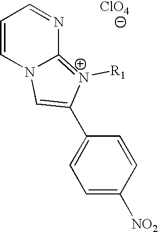
wherein R1 is an unsubstituted or at least mono-substituted linear or branched C₁₋₁₀ alkyl radical, C₂₋₁₀ alkenyl radical or C₂₋₁₀ alkynyl radical; or an unsubstituted or at least mono-substituted 6-, 10- or 14-membered aryl, which may be bonded via a linear or branched, unsubstituted or at least mono-substituted C₁₋₆ alkylene, C₂₋₆ alkenylene or C₂₋₆ alkynylene group, for the detection of spoilage in packaged raw fish, meat and poultry.
